(19) United States
(12) Patent Application Publication
(10) Pub. No.: US 2013/0253202 A1
(43) Pub. Date: Sep. 26, 2013

(54) METHOD OF TREATING HEMATOLOGICAL CANCERS

(71) Applicant: Niiki Pharma Inc., Hoboken, NJ (US)

(72) Inventors: Hooshmand Sheshbaradaran, Hoboken, NJ (US); Rebecca Baerga, Hoboken, NJ (US); Jenel Cobb, Hoboken, NJ (US)

(73) Assignee: Niiki Pharma Inc., Hoboken, NJ (US)

(21) Appl. No.: 13/896,344

(22) Filed: May 17, 2013

Related U.S. Application Data
(63) Continuation of application No. PCT/US2011/061096, filed on Nov. 17, 2011.
(60) Provisional application No. 61/414,892, filed on Nov. 17, 2010.

(51) Int. Cl. A61K 31/555

(52) U.S. Cl. CPC A61K 31/555
USPC 548/108

(57) ABSTRACT

Methods and compositions for treating hematological cancer are disclosed, including refractory or resistant hematological cancer.

### CROSS-REFERENCE TO RELATED U.S. APPLICATIONS

This is a continuation of PCT/US11/61096 filed on Nov. 17, 2011, which claims the benefit of, and priority to, U.S. Provisional Application No. 61/414,892 filed on Nov. 17, 2010, the entire content of both of which being incorporated herein by reference in their entirety.

### FIELD OF THE INVENTION

The present invention generally relates to pharmaceutical compositions and methods for treating cancer, and particularly to a method of treating hematological cancers.

### BACKGROUND OF THE INVENTION

Hematological malignancies or blood cancers are a group of diverse cancers originated from bone marrow or lymphatic tissues, affecting blood functions. Each year, new cases of leukemia, Hodgkin's and non-Hodgkin's lymphoma and myeloma account for almost 10 percent of all new cancer cases diagnosed in the United States. While targeted therapies using antibodies and kinase inhibitors (e.g., imatinib—a BCR-ABL inhibitor) have been developed, chemotherapy and radiation therapy are still heavily relied upon in the management of blood cancers. They typically exhibit significant side effect and produce low efficacy. There is a need for new classes of drugs with distinct mechanism of actions in treating blood cancers.

### SUMMARY OF THE INVENTION

The present invention provides methods of treating various hematological cancers. In one aspect, the present invention provides a method of treating, preventing or delaying the onset of, a hematological cancer (e.g., leukemia or lymphoma) comprising administering to a patient having hematological cancer a therapeutically or prophylatically effective amount of a pharmaceutically acceptable salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]).

In accordance with another aspect, a method of treating, preventing or delaying the onset of a refractory hematological cancer (e.g., leukemia or lymphoma) is provided comprising administering a therapeutically or prophylatically effective amount of a pharmaceutically acceptable salt of trans-ketrachlorobis(1H-indazole)ruthenate(III) (e.g., sodium trans-[tetrachlorobis(1H-indazole)ruthenate(III)] or potassium trans-[tetrachlorobis(1H-indazole)ruthenate(III)]) to a patient refractory to one or more drugs chosen from doxorubicin, cytarabine (Ara-C), fludarabine, melphalan, doxorubicin, cyclophosphamide, adriamycin, vincristine, and prednisone.

Use of pharmaceutically acceptable salts of trans-[etrachlorobis(1H-indazole)ruthenate(III)] for the manufacture of a medicament for use in the methods of the present invention is also provided.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF DRAWINGS

FIG. 1 is a graph showing cell viability and inhibition of proliferation of MV4-11 cells by sodium trans-[tetrachlorobis(1H-indazole)ruthenate(III)], which inhibits proliferation in a dose-dependent manner up to 100% (exposure time 24 hours). Y axis: % of control, X axis: concentration (μM).

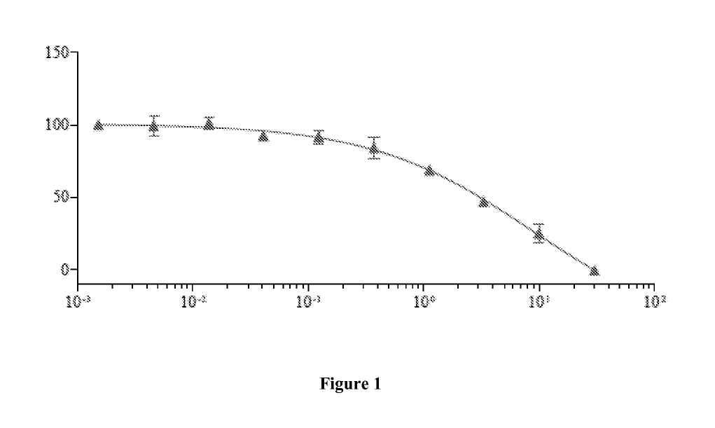

### DETAILED DESCRIPTION OF THE INVENTION

The present invention is at least in part based on the discovery that the compound sodium trans-[tetrachlorobis(1H-indazole)ruthenate(III)] has a particularly low effective IC₅₀ value in causing apoptosis in leukemia cells and lymphoma cells, and even in leukemia and lymphoma cells resistant to other chemotherapeutics. Accordingly, in accordance with a first aspect of the present invention, a method is provided for treating hematological cancers (e.g., leukemia or lymphoma). The method comprises treating a hematological cancer patient in need of treatment with a therapeutically effective amount of a pharmaceutically acceptable salt of trans-[tetrachlorobis(1H-indazole)ruthenate(III)] (e.g., alkali metal salts such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]). That is, the present invention is directed to the use of an effective amount of a pharmaceutically acceptable salt of trans-[tetrachlorobis(1H-indazole)ruthenate(III)] for the manufacture of medicaments for treating a hematological cancer (e.g., leukemia or lymphoma) in patients identified or diagnosed as having such a hematological cancer. In preferred embodiments, sodium trans-[tetrachlorobis(1H-indazole)ruthenate(III)] is used.

In one embodiment, the hematological cancer treated in accordance with the present invention is a hematological cancer of myeloid origin, i.e., derived from myeloid cells. In specific embodiments, the method of the present invention is used for treating a myelogenous leukemia. In some specific embodiments the method of the present invention is used for treating acute myelogenous leukemia (AML) or acute monoblastic/monocytic leukemia (AMOL). In some specific embodiments the method of the present invention is used for treating chronic granulocytic leukemia (CGL), chronic myelogenous leukemia (CML), myelodysplastic syndrome (MDS), or myeloproliferative disease (MPD).

In another embodiment, the hematological cancer treated in accordance with the present invention is lymphoma, a hematological cancer of lymphocyte origin, i.e., derived from lymphatic cells of the immune system. In some embodiments, the lymphoma is Hodgkin's lymphoma. In some embodiments, the lymphoma is non-Hodgkin's lymphoma. In some embodiments, the lymphoma is B cell lymphoma. In a specific embodiment, the lymphoma is diffuse large B cell lymphoma. In another specific embodiment, the lymphoma is follicular lymphoma. In another specific embodiment, the lymphoma is mantle cell lymphoma.

In the various embodiments of this aspect of the present invention, the treatment method optionally also comprises a step of diagnosing or identifying a patient as having a hematological cancers. The identified patient is then treated with or administered with a therapeutically effective amount of a compound of the present invention, e.g., sodium trans-[tetrachlorobis(1H-indazole)ruthenate(III)]. Various hematological cancers can be diagnosed in any conventional diagnostic methods known in the art including complete blood count, blood film, lymph node biopsy, bone marrow biopsy, cytogenetics analysis (e.g., for AML, CML), or immuophenotyping (e.g., for lymphoma, CLL).

In addition, it has also been surprisingly discovered that the compound sodium trans-[tetrachlorobis(1H-indazole)ruthenate(III)] is equally effective in leukemia cells resistant to doxorubicin, cytarabine (Ara-C) and fludarabine, and in lymphoma cells resistant to melphalan, doxorubicin and CHOP (cyclophosphamide, adriamycin, vincristine, and prednisone) therapy. Accordingly, another aspect of the present invention provides a method of treating leukemia comprising treating a patient identified as having leukemia previously treated with a treatment regimen comprising one or more drugs chosen from doxorubicin, cytarabine (Ara-C) and fludarabine, with a therapeutically effective amount of a pharmaceutically acceptable salt of trans-[tetrachlorobis(1H-indazole)ruthenate(III)] (e.g., alkali metal salts such as sodium trans-[tetrachlorobis(1H-indazole)ruthenate(III)] and potassium trans-[tetrachlorobis(1H-indazole)ruthenate(III)], or indazolium trans-[tetrachlorobis(1H-indazole)ruthenate(III)]). In some embodiments, the patient has leukemia that is refractory to a treatment comprising one or more drugs selected from the group consisting of doxorubicin, cytarabine (Ara-C) and fludarabine. The patient either did not respond to such a treatment regimen, or the cancer relapsed or recurred after the treatment regimen. That is, the present invention is also directed to the use of a pharmaceutically acceptable salt of trans-[tetrachlorobis(1H-indazole)ruthenate(III)] (e.g., sodium trans-[tetrachlorobis(1 H-indazole)ruthenate(III)]) for the manufacture of medicaments for treating refractory hematological cancer (e.g., AML or AMOL) refractory to one or more drugs chosen from doxorubicin, cytarabine (Ara-C) and fludarabine. In one embodiment, the refractory hematological cancer is refractory AML. In another embodiment, the refractory hematological cancer is refractory AMOL. In yet another embodiment, the refractory hematological cancer is refractory chronic myelogenous leukemia (CML). In another embodiment, the refractory hematological cancer is refractory chronic granulocytic leukemia (CGL). In another embodiment, the refractory hematological cancer is refractory myelodysplastic syndrome (MDS). In yet another embodiment, the refractory hematological cancer is refractory or myeloproliferative disease (MPD).

The present invention also provides a method of treating lymphoma comprising treating a lymphoma previously treated with a regimen comprising one or more drugs chosen from melphalan, doxorubicin, cyclophosphamide, adriamycin, vincristine, and prednisone, with a therapeutically effective amount of a pharmaceutically acceptable salt of trans-[tetrachlorobis(1H-indazole)ruthenate(III)] (e.g., alkali metal salts such as sodium trans-[tetrachlorobis(1H-indazole)ruthenate(III)] and potassium trans-[tetrachlorobis(1H-indazole)ruthenate(III)], or indazolium trans-[tetrachlorobis(1H-indazole)ruthenate(III)]). In some embodiments, the lymphoma is previously treated with the CHOP (cyclophosphamide, adriamycin, vincristine, and prednisone) therapy or RCHOP (rituximab, cyclophosphamide, adriamycin, vincristine, and prednisone) regimen.

In some embodiments, the patient has lymphoma that is refractory to a treatment comprising one or more drugs selected from the group consisting of melphalan, doxorubicin, cyclophosphamide, adriamycin, vincristine, and prednisone. The patient either did not respond to such a treatment regimen, or the cancer relapsed or recurred after the treatment regimen. That is, the present invention is also directed to the use of a pharmaceutically acceptable salt of trans-[tetrachlorobis(1H-indazole)ruthenate(III)] (e.g., sodium trans-[tetrachlorobis(1H-indazole)ruthenate(III)]) for the manufacture of medicaments for treating refractory lymphoma refractory to one or more drugs chosen from melphalan, doxorubicin, cyclophosphamide, adriamycin, vincristine, and prednisone. In one embodiment, the refractory lymphoma is refractory non-Hodgkin's lymphoma. In another embodiment, the refractory lymphoma is refractory Hodgkin's lymphoma. In a specific embodiment, the refractory lyphoma is refractory B cell lymphoma. In a specific embodiment, the refractory lyphoma is refractory diffuse large B cell lymphoma. In a specific embodiment, the refractory lyphoma is refractory follicular lymphoma. In another specific embodiment, the refractory lymphoma is refractory mantle cell lymphoma.

In a specific embodiment, the present invention also provides a method of treating lymphoma comprising treating a refractory diffuse large B cell lymphoma previously treated with a regimen comprising melphalan or doxorubicin or both, or a CHOP regimen, or a RCHOP regimen, with a therapeutically effective amount of a pharmaceutically acceptable salt of trans-[etrachlorobis(1H-indazole)ruthenate(III)] (e.g., alkali metal salts such as sodium trans-[etrachlorobis(1H-indazole)ruthenate(III)] and potassium trans-[tetrachlorobis(1H-indazole)ruthenate(III)], or indazolium trans-[tetrachlorobis(1H-indazole)ruthenate(III)]).

The term “refractory” as used herein refers to a cancer that either fails to respond favorably to an anti-neoplastic treatment that does not include a pharmaceutically acceptable salt of trans-[tetrachlorobis(1H-indazole)ruthenate(III)], or alternatively, recurs or relapses after responding favorably to an antineoplastic treatment that does not include a pharmaceutically acceptable salt of trans-[tetrachlorobis(1H-indazole)ruthenate(III)].

Thus, in some embodiments, in the method of the present invention, a pharmaceutically acceptable salt of trans-[etrachlorobis(1H-indazole)ruthenate(III)] is administered to a hematological cancer patient previously treated with a regimen comprising doxorubicin. In specific embodiments, the patient has refractory AML or AMOL, i.e., AML or AMOL that exhibits resistance to, or relapsed after, a treatment including doxorubicin. In other embodiments, a pharmaceutically acceptable salt of trans-[etrachlorobis(1H-indazole)ruthenate(III)] is administered to a hematological cancer patient previously treated with a regimen comprising cytarabine (Ara-C). In specific embodiments, the patient has refractory AML or AMOL, i.e., AML or AMOL that exhibits resistance to, or relapsed after, a treatment including cytarabine (Ara-C). In yet other embodiments, a pharmaceutically acceptable salt of trans-[tetrachlorobis(1H-indazole)ruthenate(III)] is administered to a hematological cancer patient previously treated with a regimen comprising fludarabine. In specific embodiments, the patient has refractory AML or AMOL, i.e., AML or AMOL that exhibits resistance to, or relapsed after, a treatment including fludarabine.

Thus, in some embodiments, in the method of the present invention, a pharmaceutically acceptable salt of trans-[etrachlorobis(I H-indazole)ruthenate(III)] is administered to a lymphoma (non-Hodgkin's lymphoma or Hodgkin's lymphoma) patient previously treated with a regimen comprising melphalan or doxorubicin or both, or with a CHOP or RCHOP regimen. In specific embodiments, the patient has refractory non-Hodgkin's lymphoma or Hodgkin's lymphoma that exhibits resistance to, or relapsed after, the treatment regimen. In a specific embodiment, a pharmaceutically acceptable salt of trans-[tetrachlorobis(1H-indazole)ruthenate(III)] is administered to a patient having B cell lymphoma (e.g., diffuse large B cell lymphoma, follicular lymphoma or mantle cell lymphoma) previously treated with (in particular, resistance to or relapsed after) a regimen comprising melphalan or doxorubicin or both, or with a CHOP or RCHOP regimen.

To detect a refractory hematological cancer, patients undergoing initial treatment can be carefully monitored for signs of resistance, non-responsiveness or recurring hematological cancer. This can be accomplished by monitoring the patient's cancer's response to the initial treatment. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst. 92:205-216 (2000).

In accordance with yet another aspect of the present invention, a method is provided for preventing or delaying the onset of hematological cancer, or preventing or delaying the recurrence of hematological cancer (leukemia, non-Hodgkin's lymphoma (B cell lymphoma such as diffuse large B cell lymphoma, follicular lymphoma or mantle cell lymphoma), which comprises treating a patient in need of the prevention or delay with a prophylatically effective amount of a pharmaceutically acceptable salt of trans-[tetrachlorobis(1H-indazole)ruthenate(III)] (e.g., sodium trans-[tetrachlorobis(1H-indazole)ruthenate(III)]). For purposes of preventing or delaying the recurrence of hematological cancer, hematological cancer patients who have been treated and are in remission or in a stable or progression free state may be treated with a pharmaceutically acceptable salt of trans-[tetrachlorobis(1 H-indazole)ruthenate(III)] (e.g., sodium trans-[tetrachlorobis(1H-indazole)ruthenate(III)]) to effectively prevent or delay the recurrence or relapse of hematological cancer.

As used herein, the phrase “treating . . . with . . . ” or a paraphrase thereof means administering a compound to the patient or causing the formation of a compound inside the body of the patient.

In accordance with the method of the present invention, hematological cancer can be treated with a therapeutically effective amount of a pharmaceutically acceptable salt of trans-[tetrachlorobis(1H-indazole)ruthenate(III)] alone as a single agent, or alternatively in combination with one or more other anti-cancer agents. Example of pharmaceutically acceptable salts include alkali metal salts (e.g., sodium or potassium salt), ammonium salts, indazolium salts, etc.

A pharmaceutically acceptable salt of trans-[tetrachlorobis(1H-indazole)ruthenate(III)] can be administered through intravenous injection or oral administration or any other suitable means at an amount of from 0.1 mg to 1000 mg per kg of body weight of the patient based on total body weight. The active ingredients may be administered at predetermined intervals of time, e.g., three times a day. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with the present invention, it is provided a use of a pharmaceutically acceptable salt of trans-[tetrachlorobis(1H-indazole)ruthenate(III)] (e.g., sodium trans-[tetrachlorobis(1H-indazole)ruthenate(III)]) for the manufacture of a medicament useful for treating hematological cancer. The medicament can be, e.g., in an oral or injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of a pharmaceutically acceptable salt of trans-[tetrachlorobis(1H-indazole)ruthenate(III)] (e.g., sodium trans-[tetrachlorobis(H-indazole)ruthenate(III)]), and optionally instructions for using the kit in the methods in accordance with the present invention, e.g., treating, preventing or delaying the onset of hematological cancer, or preventing or delaying the recurrence of hematological cancer, or treating refractory hematological cancer. As will be apparent to a skilled artisan, the amount of a therapeutic compound in the unit dosage form is determined by the dosage to be used on a patient in the methods of the present invention. In the kit, a pharmaceutically acceptable salt of trans-[tetrachlorobis(1H-indazole)ruthenate(III)] (e.g., sodium trans-[tetrachlorobis(1H-indazole)ruthenate(III)]) can be in a tablet form in an amount of e.g., 1 mg.

### EXAMPLE 1

### Activities of Sodium trans-[tetrachlorobis(1H-indazole)ruthenate(III)] in Human Leukemia Cells

The human leukemia cell line MV4-11 cells [biphenotypic B myelomonocytic leukemia, lymphoblast morphology] were placed in a 96-well microculture plate (Costar white, flat bottom #3917) in a total volume of 90 μL/well. After 24 hours of incubation in a humidified incubator at 37° C. with 5% CO2 and 95% air, 10 μL of 10X, serially diluted sodium trans-[tetrachlorobis(1H-indazole)ruthenate(III)] in growth medium was added to each well. After 96 total hours of culture in a CO2 incubator, the plated cells and Cell Titer-Glo (Promega #G7571) reagents were brought to room temperature to equilibrate for 30 minutes. 100 μL of Cell Titer-Glo® reagent was added to each well. The plate was shaken for 2 minutes and then left to equilibrate for 10 minutes before reading luminescence on the Tecan GENios microplate reader. Percent inhibition of cell growth was calculated relative to untreated control wells. All tests were performed in duplicate at each concentration level. The IC₅₀ value for the test agent was estimated using Prism 3.03 by curve-fitting the data using the following four parameter-logistic equation:

$$Y = \frac{Top - Bottom}{1 + {( X / {IC}_{50} )}^{n}} + Bottom$$

where Top is the maximal % of control absorbance, Bottom is the minimal % of control absorbance at the highest agent concentration, Y is the % of control absorbance, X is the agent concentration, IC50 is the concentration of agent that inhibits cell growth by 50% compared to the control cells, and n is the slope of the curve. The compound sodium trans-[tetrachlorobis(1H-indazole)ruthenate(III)] had an IC₅₀ on MV4-11 of 8.53 μM. It has been known that the MV4- 11 cells are resistant to Ara-C, fluradabine, and doxorubicin. See Colado et al., Haematologica., 93(1):57-66 (2008); Scatena et al., Cancer Chemother. Pharmacol., 66(5):881-8 (2010).

### EXAMPLE 2

### Activities of Sodium trans-[tetrachlorobis(1H-indazole)ruthenate(III)] in Human Lymphoma Cells

To determine the antiproliferative activity of sodium trans-[tetrachlorobis(1H-indazole)ruthenate(III)] in human lymphoma tumor cell lines, anti-proliferation assays were conducted in the DoHH2 (follicular lymphoma), Granta 519 (mantle cell lymphoma), and WSU-DLCL2 (diffuse large B cell lymphoma) cell lines. The DoHH2 Human EBV-negative B Cell Lymphoma cells were seeded with 5,000 cells/well and grown in RPMI1640 medium containing 20% FBS, and 2mM L-Glutamine. The Granta 519 Human Mantle Cell Lymphoma cells were seeded with 10,000 cells/well and grown in DMEM medium containing 10% FBS, and 2 mM L-Glutamine. The WSU-DLCL2 Human B Cell Lymphoma cells were seeded with 5,000 cells/well and grown in RPMI1640 medium containing 10% FBS, and 2mM L-Glutamine. Specifically, the human tumor cells were placed in a 96-well microculture plate at the appropriate density for 96 hours of total growth time. After 24 hours of incubation in a humidified incubator at 37° C. with 5% CO₂ and 95% air, serially diluted test agents in growth medium were added to each well. After 96 total hours of culture in a CO₂ incubator, the plates were processed with Cell Titer-Glo (Promega #G7571) according to manufacturer's instructions. Luminescence was detected using a Tecan GENios microplate reader. Percent inhibition of cell growth was calculated relative to untreated control wells. All tests were performed in duplicate at each concentration level.

The IC₅₀ value for the test agents was estimated using Prism 3.03 by curve-fitting the data using the following four parameter-logistic equation:

$$Y = \frac{Top - Bottom}{1 + {( X / {IC}_{50} )}^{n}} + Bottom$$

where Top is the maximal % of control absorbance, Bottom is the minimal % of control absorbance at the highest agent concentration, Y is the % of control absorbance, X is the agent concentration, IC50 is the concentration of agent that inhibits cell growth by 50% compared to the control cells, and n is the slope of the curve. The compound sodium trans-[tetrachlorobis(1H-indazole)ruthenate(III)] had an IC₅₀ of 7.18 μM in DoHH2, 63.56 μM in Granta 519, and 25 μM in WSU-DLCL2. WSU-DLCL2 cells are resistant to melphalan and doxorubicin, and also resistant to CHOP (cyclophosphamide, adriamycin, vincristine, and prednisone) therapy. See Aref et al., Int. J. Radiation Oncology Biol. Phys., 45(4): 999-1003 (1999); Levi et al., Cancer Chemother. Pharmacol Published on line Aug. 31, 2010. Thus, sodium trans-[tetrachlorobis(1H-indazole)ruthenate(III)] can be effective in cancer cell resistant to such drugs.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. Use of a compound that is a pharmaceutically acceptable salt of trans-[tetrachlorobis(1H-indazole)ruthenate(III)] for the manufacturing of a medicament for treating hematological cancer.

2. The use of claim 1, wherein said hematological cancer is lymphoma.

3. The use of claim 2, wherein said lymphoma is B cell lymphoma.

4. The use of claim 3, wherein said lymphoma is diffuse large B cell lymphoma, follicular lymphoma or mantle cell lymphoma.

5. The use of claim 1, wherein said hematological cancer is a refractory hematological cancer.

6. The use of claim 5, wherein said hematological cancer is leukemia refractory to a treatment comprising one or more drugs selected from the group consisting of doxorubicin, cytarabine and fludarabine.

7. The use of claim 5, wherein said hematological cancer is lymphoma refractory to a regimen comprising doxorubicin or melphalan or both, or a CHOP regimen or a RCHOP regimen.

8. The use of claim 1, wherein said compound is sodium trans-[tetrachlorobis(1H-indazole)ruthenate(III)].
